(19) United States
(12) Patent Application Publication
(10) Pub. No.: US 2024/0335143 A1
(43) Pub. Date: Oct. 10, 2024

(54) NON-INVASIVE GLUCOSE MONITORING MODULE WITH POLARIZED LIGHT

(71) Applicant: Taiwan-Asia Semiconductor Corporation, Hsinchu City (TW)

(72) Inventors: Tzu-Hui LIN, Hsinchu City (TW); Sheng-Wei CHEN, Hsinchu City (TW)

(21) Appl. No.: 18/629,316

(22) Filed: Apr. 8, 2024

(30) Foreign Application Priority Data
Apr. 8, 2023 (TW) 112113188

(51) Int. Cl. A61B 5/145; A61B 5/1455

(52) U.S. Cl. CPC A61B 5/14532; A61B 5/14558; A61B 2562/0238

(57) ABSTRACT

The invention provides a non-invasive blood glucose monitoring module with polarized light, which comprises a light emitting component, a light receiving component and a connecting member. The light emitting component includes a light emitting element and a first polarizing element, and light emitted from the light emitting element passes through the first polarizing element to transform into polarized light. A light emitting surface is formed for the light emitting component. The light receiving component includes a light sensing element, a magnetic crystal layer and a second polarizing element, and the light sensing element receives the polarized light being reflected and sequentially passing through the magnetic crystal layer and the second polarizing element. A light receiving surface is formed for the light receiving component. The connecting member connects one side of the light emitting component and one side of the light receiving component.

### CROSS REFERENCE TO RELATED APPLICATION

This application claims the benefit of Taiwan Patent Application Serial No. 112113188 filed on Apr. 8, 2023. The entirety of each application is incorporated herein by reference.

### BACKGROUND OF THE INVENTION

### 1. Field of the Invention

The present invention relates to a blood glucose monitoring module, especially a non-invasive blood glucose monitoring module utilizing optical components for polarized light.

### 2. Description of Related Art

Structural designs of non-invasive blood glucose monitoring modules alter based on different light sources applied. In recent years, light sources that can emit polarized light have been used in non-invasive blood glucose monitoring modules. Conventional glucose monitoring modules with polarized light generally use He—Ne lasers as sensing light sources, with photoelectric modulators or magnetic cavities composed of magnets, to process corresponding signal modulation for performing blood glucose monitoring operations. However, whether for generation devices of He—Ne laser, photoelectric modulators or magnetic cavities, bulky optical or electronic components should be cooperatively arranged, and the corresponding structural designs are relatively complex, causing that the whole non-invasive blood glucose monitoring modules are unable to meet demands for miniaturization. Thus, it is difficult for the conventional non-invasive blood glucose monitoring modules with polarized light to be applied to wearable devices. Therefore, the conventional non-invasive blood glucose monitoring modules with polarized light are subject to many limitations in sensing applications, which affects their flexibility of use.

In light of this, it is really worthy of research and development, for solving those above-mentioned problems, to design a non-invasive blood glucose monitoring module with polarized light for applying to wearable devices.

### SUMMARY OF THE INVENTION

An objective of the present invention is to provide a non-invasive blood glucose monitoring module with polarized light.

To achieve the above mentioned objective, the non-invasive blood glucose monitoring module with polarized light of the present invention comprises a light emitting component, a light receiving component and a connecting member. The light emitting component includes a light emitting element and a first polarizing element, and light emitted from the light emitting element passes through the first polarizing element to transform into polarized light. A light emitting surface is formed for the light emitting component. The light receiving component includes a light sensing element, a magnetic crystal layer and a second polarizing element, and the light sensing element receives the polarized light being reflected and sequentially passing through the magnetic crystal layer and the second polarizing element. A light receiving surface is formed for the light receiving component. The connecting member connects one side of the light emitting component and one side of the light receiving component.

In one embodiment of the present invention, one end of the connecting member, adjacent to the light emitting surface and the light receiving surface, forms an included angle A, and the included angle A is between 0 and 90 degrees.

In one embodiment of the present invention, the connecting member is a trapezoidal prism, a triangular prism or a sector-shaped prism.

In one embodiment of the present invention, a wavelength range corresponding to the light emitting element is between 500 nm and 1800 nm.

In one embodiment of the present invention, the light emitting element is a vertical cavity surface emitting laser or a light emitting diode.

In one embodiment of the present invention, the magnetic crystal layer is made from materials that can produce a magnetization phenomenon induced under influencing of an external magnetic field or that contain rare earth elements.

In one embodiment of the present invention, the first polarizing element and the second polarizing element are made from polymers or metals.

In one embodiment of the present invention, the light emitting component further includes a first cover, and the first cover is disposed on one side, facing away from the light emitting element, of the first polarizing element. The light receiving component further includes a second cover, and the second cover is disposed on one side, facing away from the light sensing element, of the magnetic crystal layer.

In one embodiment of the present invention, the first cover and the second cover are made of glass.

In one embodiment of the present invention, the light emitting component further includes a first substrate and a first light shielding structure, the light emitting element is disposed on the first substrate, and the first light shielding structure is sandwiched between the first substrate and the first polarizing element. The light receiving component further includes a second substrate and a second light shielding structure, the light sensing element is disposed on the second substrate, and the second light shielding structure is sandwiched between the second substrate and the second polarizing element.

In one embodiment of the present invention, the first light shielding structure and the second light shielding structure are made from non-transparent epoxy resins.

Hereby, by applying a miniaturized structure design to generate polarized light for blood glucose monitoring, the non-invasive blood glucose monitoring module with polarized light of the present invention is convenient for application in wearable devices and has higher flexibility of use. On the other hand, through altering the relative angle between the light emitting component and the light receiving component by the connecting member, the light signal received by the light receiving component is maximized, so as to optimize performance of blood glucose monitoring.

### BRIEF DESCRIPTION OF THE DRAWINGS

FIG. 1 is a schematic view of a first embodiment of a non-invasive blood glucose monitoring module with polarized light according to the present invention; and

FIG. 2 is a schematic view of a second embodiment of the non-invasive blood glucose monitoring module with polarized light according to the present invention.

### DETAILED DESCRIPTION OF THE PREFERRED EMBODIMENT

Since various examples and embodiments in the present invention are only illustrative and non-restrictive, a person skilled in the art can easily conceive other examples and embodiments without contravening the scope of the present invention, after reading this specification, and can make the features and advantages of these embodiments more evident based on the following detailed description and claims.

Herein, the description of unit, element and component in the present invention uses “one”, “a”, or “an”. This is for convenience and for offering general meaning of the category of the present invention. Therefore, the description should be understood as including “one”, “at least one”, and singular and plural forms at the same time unless the context clearly indicates otherwise.

Herein, the description of the terms “first” or “second” and similar ordinal numbers are mainly used to distinguish or refer to the same or similar elements or structures and do not necessarily imply that such components or structures are spatially or temporally distinct order. It should be understood that ordinal numbers, in certain situations or configurations, may be used interchangeably without affecting the implementation of the present invention.

Herein, the description of “comprise”, “have” or other similar semantics have the non-exclusive meaning. For example, components or structures with a plurality of elements are not only limited to those disclosed in this specification, but also include generally inherent elements, which are not explicitly listed here for the components or the structures.

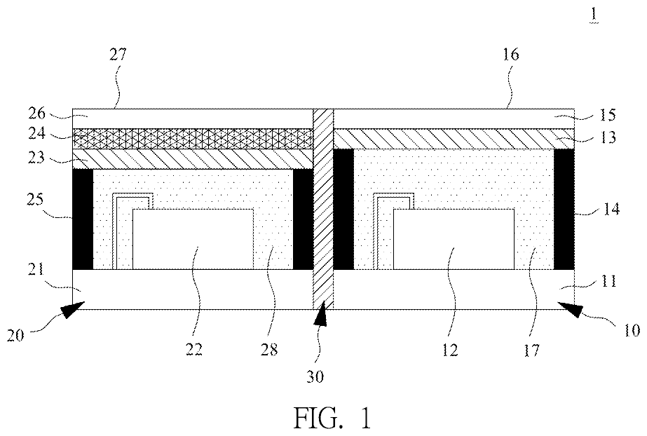

Please refer to FIG. 1, which is a schematic diagram of a first embodiment of a non-invasive blood glucose monitoring module with polarized light according to the present invention. As shown in FIG. 1, the non-invasive blood glucose monitoring module with polarized light 1 comprises a light emitting component 10, a light receiving component 20 and a connecting member 30. The light emitting component 10 mainly generates polarized light for exposing to skins. The light receiving component 20 mainly receives the polarized light that is diffusely reflected by the skins and then transmitted back. The connecting member 30 is utilized to connect the light emitting component 10 and the light receiving component 20.

The light emitting component 10 includes a first substrate 11, a light emitting element 12 and a first polarizing element 13. The first substrate 11 serves as a basic structure of the light emitting component 10, and the first substrate 11 is adopted to carry the light emitting element 12, the first polarizing element 13 and other related elements. The light emitting element 12 is disposed on the first substrate 11 and serves as a light source to emit light. The light emitting element 12 can be electrically connected to the first substrate 11 through metal wires. In the present invention, light emitting element 12 is a laser diode, such as a vertical cavity surface emitting laser (VCSEL), but not limited thereto. For example, a light emitting diode (LED) or other types of light sources may also be used for the light emitting element 12. A wavelength range corresponding to the light emitting element 12 is approximately between 500 nm and 1800 nm. For example, in one embodiment of the present invention, the wavelength corresponding to the light emitting element 12 is 650 nm. The first polarizing element 13 is disposed in an emission path of the light emitted by the light emitting element 12, such as directly disposed above the light emitting element 12, so that the light passes through the first polarizing element 13 to transform into linearly polarized light. In the present invention, the first polarizing element 13 is made from polymers or metals, but the invention is not limited thereto.

In one embodiment of the present invention, the light emitting component 10 further includes a first light shielding structure 14. The first light shielding structure 14 is sandwiched between the first substrate) 11 and the first polarizing element 13, and the first light shielding structure 14 is mainly arranged around the light emitting element 12. The first light shielding structure 14 is principally applied to stop the light emitted by the light emitting element 12 from being laterally scattered, so that the light can be intensively emitted toward the first polarizing element 13. The first light shielding structure 14 is made from non-transparent epoxy resins, such as a packaging black-glue, but the invention is not limited thereto.

In one embodiment of the present invention, the light emitting component 10 further includes a first cover 15. The first cover 15 is stacked on the first polarizing element 13, and one side of the first cover 15 facing away from the first polarizing element 13 (namely, a side facing the skins) forms a light emitting surface 16 of the light emitting component 10. The first cover 15 serves as a protection member of the light emitting component 10, and the first cover 15 is made of light-permeable glass, but the invention is not limited thereto.

In addition, a first packaging structure 17 of the light emitting component 10 is further formed by filling packaging materials between the first substrate 11, the first polarizing element 13 and the first light shielding structure 14. The first packaging structure 17 is applied for packaging and fixing the light emitting element 12 and related metal wires. The first packaging structure 17 is made from light-permeable epoxy resins, but the invention is not limited thereto.

The light receiving component 20 includes a second substrate 21, a light sensing element 22, a second polarizing element 23 and a magnetic crystal layer 24. The second substrate 21 serves as a basic structure of the light receiving component 20, and the second substrate 21 is adopted to carry the light sensing element 22, the second polarizing element 23, the magnetic crystal layer 24 and other related elements. The light sensing element 22 is disposed on the second substrate 21, and the light sensing element 22 is utilized to receive reflected light and to proceed subsequent sensing. The light sensing element 22 can be electrically connected to the second substrate 21 through metal wires. In the present invention, a photo detector is used for the light sensing element 22, but not limited thereto. The second polarizing element 23 is disposed in the incident path of the reflected light received by the light sensing element 22, for example, directly disposed above the light sensing element 22. In the present invention, the second polarizing element 23 is made from polymers or metals, but not limited thereto. The magnetic crystal layer 24 is stacked on the second polarizing element 23 and also located in the incident path of the reflected light received by the light sensing element 22, such as directly located above the light sensing element 22, so that the reflected light sequentially passes through the magnetic crystal layer 24 and the second polarizing element 23 and then is received by the light sensing element 22. The magnetic crystal layer 24 is mainly used to alter the polarization angle of the reflected light, and crystals of the magnetic crystal layer 24 can be rotated by an external magnetic field (such as an external coil or magnetic object). Therefore, the magnetic crystal layer 24 is made from materials that can produce a magnetization phenomenon induced under influencing of an external magnetic field or made from materials that contain rare earth elements. In the present invention, magneto-optic Faraday rotator garnet crystals are utilized for the magnetic crystal layer 24 as rotating crystals of the polarization angle of the reflected light. But the magnetic crystal layer 24 can also use other applicable crystals controlled by the variable magnetic field to rotate the polarization angle of the incident light.

In one embodiment of the present invention, the light receiving component 20 further includes a second light shielding structure 25. The second light shielding structure 25 is sandwiched between the second substrate 21 and the second polarizing element 23, and the second light shielding structure 25 is mainly disposed around the light sensing element 22. The second light shielding structure 25 is mainly used to stop the reflected light, which sequentially passes through the magnetic crystal layer 24 and the second polarizing element 23, from being laterally scattered, so that the reflected light can be intensively received by the light sensing element 22. The second light shielding structure 25 is made from non-transparent epoxy resins, such as a packaging black-glue, but the invention is not limited thereto.

In one embodiment of the present invention, the light receiving component 20 further includes a second cover 26. The second cover 26 is stacked on the magnetic crystal layer 24, and one side of the second cover 26 facing away from the magnetic crystal layer 24 (namely, a side facing the skins) forms a light receiving surface 27 of the light receiving component 20. The second cover 26 serves as a protection member of the light receiving component 20, and the second cover 26 is made of light-permeable glass, but not limited thereto.

In addition, a second packaging structure 28 of the light receiving component 20 is further formed by filling the packaging materials between the second substrate 21, the second polarizing element 23 and the second light shielding structure 25. The second packaging structure 28 is applied for packaging and fixing the light sensing element 22 and related metal wires. The second packaging structure 28 is made from light-permeable epoxy resins, but the invention is not limited thereto.

The connecting member 30 is disposed between the light emitting component 10 and the light receiving component 20. By that the connecting member 30 connects one side of the light emitting component 10 and one side of the light receiving component 20, the light emitting component 10, the light receiving component 20 and the connecting member 30 form an integral structure. The connecting member 30 is made of plastic, and the connecting member 30 connects the light emitting component 10 and the light receiving component 20 through an adhesive material (such as an adhesive tape), but the invention is not limited thereto. In this embodiment, the connecting member 30 is a rectangular prism, and the light emitting surface 16 of the light emitting component 10 and the light receiving surface 27 of the light receiving component 20 are located on the same plane.

In actual practice, the non-invasive blood glucose monitoring module with polarized light 1 of the present invention first emits the light by the light emitting element 12 of the light emitting component 10. The light will pass through the first polarizing element 13 and the first cover 15 in sequence, and then reaches the skins to proceed corresponding blood glucose sensing. Afterwards, the light diffusely reflected from the skins will pass through the second cover 26, the magnetic crystal layer 24 and the second polarizing element 23 in sequence, and then be received by the light sensing element 22 to proceed subsequent signal analyses of blood glucose sensing.

Since the non-invasive blood glucose monitoring module with polarized light 1 of the present invention utilizes VCSEL as its light source, which has a smaller beam divergence angle and higher energy, a signal-to-noise ratio is improved. Moreover, with arranging of the structure for generating polarized light, the overall volume of the non-invasive blood glucose monitoring module with polarized light 1 of the present invention can meet requirements for miniaturization, so as to be applied to wearable devices and to perform blood glucose monitoring on a skin surface of any part of a body.

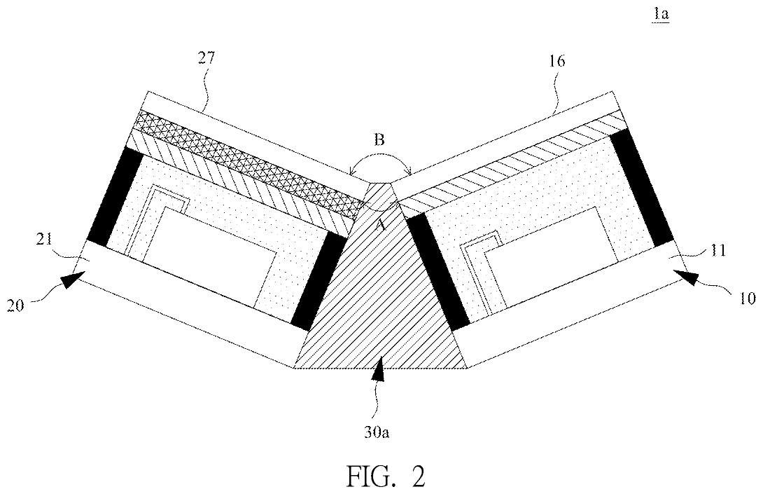

Please refer to FIG. 2, which is a schematic diagram of a second embodiment of a non-invasive blood glucose monitoring module with polarized light according to the present invention. This embodiment is a modification of the first embodiment, mainly by varying the structure of the connecting member. As shown in FIG. 2, in this embodiment, one end of the connecting member 30a of the non-invasive blood glucose monitoring module with polarized light 1a of the present invention, adjacent to the light emitting surface 16 of the light emitting component 10 and the light receiving surface 27 of the light receiving component 20, forms an included angle A, and the included angle A is between 0 and 90 degrees. That is to say, by setting the included angle A of the connecting member 30a, a corresponding angle B between 180 and 90 degrees presents between the light emitting surface 16 of the light emitting component 10 and the light receiving surface 27 of the light receiving component 20. For example, in one embodiment of the present invention, the included angle A of the connecting member 30a is about 45 degrees, so that the corresponding angle B of about 135 degrees presents between the light emitting surface 16 and the light receiving surface 27, but the invention is not limited thereto. In order to form the aforementioned included angle A, in this embodiment, the connecting member 30a may be a trapezoidal prism, a triangular prism or a sector-shaped prism.

Accordingly, in the present invention, a light emitting direction of the light emitting component 10 and a light receiving direction of the light receiving component 20 are changed through the connecting member 30a of the non-invasive blood glucose monitoring module with polarized light 1a, and an intensity of the light received by the light receiving component 20 increases, so as to optimize the performance of blood glucose monitoring.

The above implementations are only auxiliary descriptions, and are not intended to limit the embodiments of the application subject or the applications or uses of the embodiments. In addition, although at least one illustrative example has been presented above, it should be understood that the present invention can still have a large number of variations. It should also be understood that the embodiments described herein are not intended to limit the scope, use, or configuration of the requested subject matter in any way. On the contrary, the foregoing embodiments will provide a convenient guide for those skilled in the art to implement one or more embodiments. Furthermore, various changes can be made to the function and arrangement of the components without departing from the scope defined by the patent claims, and the scope of the patent claims includes known equivalents and all foreseeable equivalents at the time that the patent application is filed.

What is claimed is:

1. A non-invasive blood glucose monitoring module with polarized light, comprising:
a light emitting component, including a light emitting element and a first polarizing element, wherein light emitted from the light emitting element passes through the first polarizing element to transform into polarized light, and a light emitting surface is formed for the light emitting component,
a light receiving component, including a light sensing element, a magnetic crystal layer and a second polarizing element, wherein the light sensing element receives the polarized light being reflected and sequentially passing through the magnetic crystal layer and the second polarizing element, and a light receiving surface is formed for the light receiving component, and
a connecting member, connecting one side of the light emitting component and one side of the light receiving component.

2. The non-invasive blood glucose monitoring module with polarized light as claimed in claim 1, wherein one end of the connecting member, adjacent to the light emitting surface and the light receiving surface, forms an included angle A, and the included angle A is between 0 and 90 degrees.

3. The non-invasive blood glucose monitoring module with polarized light as claimed in claim 2, wherein the connecting member is a trapezoidal prism, a triangular prism or a sector-shaped prism.

4. The non-invasive blood glucose monitoring module with polarized light as claimed in claim 1, wherein a wavelength range corresponding to the light emitting element is between 500 nm and 1800 nm.

5. The non-invasive blood glucose monitoring module with polarized light as claimed in claim 4, wherein the light emitting element is a vertical cavity surface emitting laser or a light emitting diode.

6. The non-invasive blood glucose monitoring module with polarized light as claimed in claim 1, wherein the magnetic crystal layer is made from materials that can produce a magnetization phenomenon induced under influencing of an external magnetic field or that contain rare earth elements.

7. The non-invasive blood glucose monitoring module with polarized light as claimed in claim 1, wherein the first polarizing element and the second polarizing element are made from polymers or metals.

8. The non-invasive blood glucose monitoring module with polarized light as claimed in claim 1, wherein. the light emitting component further includes a first cover, and the first cover is disposed on one side, facing away from the light emitting element, of the first polarizing element, and wherein the light receiving component further includes a second cover, and the second cover is disposed on one side, facing away from the light sensing element, of the magnetic crystal layer.

9. The non-invasive blood glucose monitoring module with polarized light as claimed in claim 8, wherein the first cover and the second cover are made of glass.

10. The non-invasive blood glucose monitoring module with polarized light as claimed in claim 1, wherein the light emitting component further includes a first substrate and a first light shielding structure, the light emitting element is disposed on the first substrate, and the first light shielding structure is sandwiched between the first substrate and the first polarizing element, and wherein the light receiving component further includes a second substrate and a second light shielding structure, the light sensing element is disposed on the second substrate, and the second light shielding structure is sandwiched between the second substrate and the second polarizing element.

11. The non-invasive blood glucose monitoring module with polarized light as claimed in claim 10, wherein the first light shielding structure and the second light shielding structure are made from non-transparent epoxy resins.
